(19) United States
(12) Patent Application Publication
(10) Pub. No.: US 2005/0177143 A1
(43) Pub. Date: Aug. 11, 2005

(54) Remotely-controlled ablation of surfaces

(76) Inventors: Jeff Bullington, Chuluota, FL (US); Richard Stoltz, Plano, TX (US); Peter J. Delfyett, Oviedo, FL (US)

Correspondence Address: CHALKER FLORES, LLP, 12700 PARK CENTRAL, STE. 455, DALLAS, TX, 75251, US

(21) Appl. No.: 10/916,364

(22) Filed: Aug. 11, 2004

Related U.S. Application Data
(60) Provisional application No. 60/494,276, filed on Aug. 11, 2003.
(60) Provisional application No. 60/494,180, filed on Aug. 11, 2003.

(51) Int. Cl. A61B 18/20

(52) U.S. Cl. 606/10; 606/11

(57) ABSTRACT

The present invention includes an apparatus and the method for remotely controlling an ablation beam for removal of material from a body so that it to impinge at various locations on the surface and can be remotely blocked or enabled, when monitored with a video camera to view the body surface in an isolated environment.

### CROSS-REFERENCE TO RELATED APPLICATIONS

This application claims priority to U.S. Provisional Patent Applications Ser. No. 60/494,276; entitled “Remotely-Controlled Ablation of Surfaces” filed Aug. 11, 2003 (Docket No.ABI-17) and U.S. Provisional Patent Applications Ser. No. 60/494,180; entitled “Ablation Of A Custom Shaped Area” filed Aug. 11, 2003 (Docket No.ABI-18).

### TECHNICAL FIELD OF THE INVENTION

The present invention relates in general to the field of light amplification and, more particularly to remotely controlled ablation of surfaces.

### BACKGROUND OF THE INVENTION

Ablative material removal is especially useful for medical purposes, either in-vivo or on the outside surface (e.g., skin or tooth). Ablative removal of material is generally done with a short optical pulse that is stretched amplified and then compressed. A number of types of laser amplifiers have been used for the amplification.

Laser ablation is very efficiently done with a beam of short pulses (generally a pulse-duration of three picoseconds or less). While some laser machining melts portions of the work-piece, this type of material removal is ablative, disassociating the surface atoms. Techniques for generating these ultra-short pulses are described, e.g., in a book entitled “Femtosecond Laser Pulses” (C. Rulliere editor), published 1998, Springer-Verlag Berlin Heidelberg New York. Generally large systems, such as Ti:Sapphire, are used for generating ultra-short pulses (USP).

USP phenomenon was first observed in the 1970's, when it was discovered that mode-locking a broad-spectrum laser could produce ultra-short pulses. The minimum pulse duration attainable is limited by the bandwidth of the gain medium, which is inversely proportional to this minimal or Fourier-transform-limited pulse duration. Mode-locked pulses are typically very short and will spread (i.e., undergo temporal dispersion) as they traverse any medium. Subsequent pulse-compression techniques are often used to obtain USP's. Pulse dispersion can occur within the laser cavity so that compression techniques are sometimes added intra-cavity. When high-power pulses are desired, they are intentionally lengthened before amplification to avoid internal component optical damage. This is referred to as “Chirped Pulse Amplification” (CPA). The pulse is subsequently compressed to obtain a high peak power (pulse-energy amplification and pulse-duration compression).

### SUMMARY OF THE INVENTION

Ablative material removal with short optical pulses is especially useful for medical purposes, as it is almost non-thermal and generally painless. One embodiment includes, a remotely-controlled ablation beam using a video camera and a monitor-containing control module, or the system may direct the beam control without human intervention. Other embodiment includes a video camera which may be IR or UV and/or visible. It can provide for situations where, for safety reasons, the patient (or the operator) needs to be in an isolated environment (e.g., contagious diseases, radiation, or the body surface needs special illumination such as when IR or UV is used). One embodiment provides magnification. One embodiment includes special illumination conditions and greater safety of operating personnel. In one embodiment, the illumination conditions include darkened conditions for an IR camera that senses local variations in body temperature.

In one embodiment, a operator located remotely uses a computer mouse to control the ablation. However, those skilled in the art will realize other controls can be used including a joy-stick, or a touch screen, or tablet. In one embodiment, the system enables or blocks the emission of an ablation beam based on colors of the target area. In one embodiment, the system enables ablation within a range of distance from a surface. In another embodiment, the system ablates a custom-shaped area. In one embodiment, an operator may use a computer mouse, or a touch screen, to outline an area and/or select a color on a computer monitor, to set the ablation specification into the control system.

In one embodiment, the ablation-probe may be mounted on a control-system positionable table. In one embodiment, a beam-emitting probe mounted on x-y table with a system focused lens, or x-y-z-positioner. One embodiment allows an operator to direct ablation beam movement, however other embodiments can use preset conditions to allow the system to control the beam.

In one embodiment, the distance from the table to the surface is measures sonically, or by measuring backpressure from air-jet (or air-jet and suction combination). Additionally, other embodiments measure a dimension of size of an auxiliary light beam on the surface as an indication of distance. In one embodiment, the auxiliary light beam is conical. In another embodiment, the auxiliary light beam may change color when the beam is enabled. With the auxiliary beam coming from a point and scanned through an angle, the auxiliary light-beam length varies as a function of the distance from the object, and thus measuring the length of the scan's trace on the surface indicates distance. Similarly, with a conical beam, measuring the diameter of a circle on a perpendicular surface give an indication of distance (as will measuring the largest dimension of an ellipse on a slanted surface).

In one embodiment, the ablation probe is mounted on a positioner other than an x-y or x-y-z-positioner. In one embodiment, the positioner is a robotic arm, a prosthetic arm, a beam, a tray, a cart, a desk, or a vehicle. In one embodiment, moving the beam without moving the probe scans a smaller ablation area. In one embodiment, large areas are ablated by scanning the beam from a stationary probe over a first area, and then stepping the probe to second portion of the large area and then scanning the beam over the second area, and so on.

One embodiment includes a method of controlling a surgical ablation beam for ablation of a body surface in an isolated environment, including positioning a video camera to view the body surface in an isolated environment where the body surface needs to be in an isolated environment because of contagious diseases, radiation, or special illumination control; providing a remotely-controllable ablation beam that can be controlled to impinge at various locations on the surface (and may be remotely blocked or enabled); and controlling the beam using a monitor in a monitor-containing control module.

One embodiment includes a method of controlling an ablation beam for ablation of a surface (e.g., in an environment isolated for safety and/or illumination reasons) including positioning a video camera to view the body surface in an isolated environment where the surface needs to be in an isolated environment; providing a remotely controllable ablation beam that can be controlled to impinge at various locations on the surface; and controlling the beam using a monitor in a monitor-containing control module. In one embodiment, the beam is remotely blocked or enabled. The surface can include surfaces that have been exposed by previous ablation, and thus the ablation can cut into or thru an object.

One embodiment includes a method of controlling an ablation beam for ablation of a surface, including positioning at least one sensor to view the surface and sending a signal from the sensor to a remote control module; providing a remotely-controllable ablation beam that can be controlled to impinge optical pulses of ten picoseconds or less duration at various locations on the surface; and controlling location where the beam impinges the surface using a control module based on the signal received from the at least one sensor. One embodiment, controls the beam impingement by controlled an x-y table, which can also have a z-drive, or which can have a beam that passes through a variable-focus lens on the x-y table and the control module automatically adjusts the lens for changes in distance between the surface and the table. In one embodiment, the control module directs the ablation over a custom-shaped area.

One embodiment includes a method of controlling an ablation beam in the ablation of an object, includes positioning at least one sensor to view the object; providing a remotely-controllable ablation beam that can be controlled to impinge optical pulses ten picoseconds or less in duration at various locations on the object; and controlling the beam impingement using a control module that receives a signal from the at least one sensor, wherein the control module is remote from the object. In one embodiment, the control module may direct the ablation over a custom-shaped area.

### DETAILED DESCRIPTION OF THE INVENTION

While the making and using of various embodiments of the present invention are discussed in detail below, it should be appreciated that the present invention provides many applicable inventive concepts that can be embodied in a wide variety of specific contexts. The specific embodiments discussed herein are merely illustrative of specific ways to make and use the invention and do not delimit the scope of the invention.

To facilitate the understanding of this invention, a number of terms are defined below. Terms defined herein have meanings as commonly understood by a person of ordinary skill in the areas relevant to the present invention. Terms such as “a”, “an” and “the” are not intended to refer to only a singular entity, but include the general class of which a specific example may be used for illustration. The terminology herein is used to describe specific embodiments of the invention, but their usage does not delimit the invention, except as outlined in the claims.

Ablative material removal with short optical pulses is especially useful for medical purposes, as it is almost non-thermal and generally painless. In one embodiment, the system includes a remotely controlled ablation beam using a video camera and a monitor containing control module. In other embodiments the camera is IR or UV and/or visible. In one embodiment, the system is used in situations where the patient is isolated for example: contagious diseases, radiation, or patient needs special environment. Other situations include uses where magnification is needed, or IR or UV used. It can provide for greater safety of operating personnel. In one embodiment, an operator uses a computer touch screen to control the ablation. In another embodiment, an operator uses a mouse to direct ablation, or designate an area to be ablated.

In one embodiment, the ablation-probe may be mounted on a control-system positionable table. In another embodiment, a beam-emitting probe mounted on x-y table with a system focused lens, or x-y-z-positioner. In one embodiment, an operator directs the ablation beam movement. In another embodiment, the system control directs the beam using preset conditions.

In one embodiment, a control module automatically adjusts a lens (through which the beam passes) to focus the beam depending on a measured distance from the table to the surface to be ablated. In another embodiment, the ablation probe is mounted on an x-y-z-positioner, and the control system moves the probe in the z-direction to follow surfaces that are not flat.

In one embodiment, the measurement of distance from the table to the surface is made sonically, or by measuring backpressure from air-jet (or suction, or air-jet and suction combination). In another embodiment, the measurement is made by measuring a dimension of size of an auxiliary light beam on the surface as an indication of distance. In one embodiment, the auxiliary light beam may be conical. With a beam coming from a point and scanned through an angle, the auxiliary light-beam length varies as a function of the distance from the object, and thus measuring the length of the scan's trace on the surface indicates distance. One embodiment includes a conical beam that measuring the diameter of a circle on a perpendicular surface give an indication of distance, as will measuring the largest dimension of an ellipse on a slanted surface.

In one embodiment, the ablation probe is mounted on a positioner other than an x-y or x-y-z-positioner. In another embodiment, the positioner is a robotic arm, a prosthetic arm, a beam, a tray, a cart, a desk, or a vehicle. In one embodiment, moving the beam without moving the probe scans a smaller ablation area. In another embodiment, the auxiliary light beam may change color when the beam is on.

In one embodiment, an operator uses a variety of input devices including a computer mouse or keyboard, joy-stick, or touch screen, or tablet to select an ablation area using the control module monitor. In another embodiment, the system is enables or blocks the emission of an ablation beam based on colors of the target area and/or enable ablation within a range of distance from a surface. In another embodiment, a surgeon uses the same input device to select color and area on a monitor to set the specifications into the control system.

One embodiment includes a method of controlling a surgical ablation beam for ablation of a body surface, including positioning a video camera to view the body surface; providing a video display monitor for the video camera in a monitor-containing control module; selecting at least one area shown on the monitor for ablation, and inputting information on the selected area into a control module; providing an ablation beam that can be electrically and/or mechanically controlled to impinge at various locations on the surface; and controlling the beam using the control module in ablation of the surface. In another embodiment, the beam-emitting probe is not manually moved to change the location of impingement.

Another embodiment includes a method of controlling an ablation beam for ablation of a body surface, including positioning a video camera to view the body surface; providing a video display from the video camera on a monitor of a control module; selecting at least one ablation area on the monitor, and inputting selected area information into the control module; providing an ablation beam that can be controlled to impinge at various locations on the surface; and using the control module to control the beam in ablation of the surface.

In another embodiment, the system controls the emission of an ablation beam based on colors of the target area (see “Enabling Or Blocking The Emission Of An Ablation Beam Based On Color Of Target Area” provisional application Docket No.ABI-16; which is incorporated by reference herein). In one embodiment, a surgeon uses a computer mouse, or a touch screen, to select a color on a computer monitor to set the color specification into the control system. In one embodiment, a surgeon directs ablation beam movement. In another embodiment, conditions are preset and the system controls the beam. One embodiment scans over a larger area with ablation only enabled for specific color to be removed or a mole. In one embodiment, the color is in a tattoo, in paint, in a poster, wall, vehicle, floor or cloth. In one embodiment, the emission blocked where a colored marker cream had been placed on a surface indicating where a surgeon does not want to ablate. In another embodiment, the cream shields skin from unwanted ablation. In one embodiment, the color is red, blue, green, yellow or combination thereof. In one embodiment, the surface is skin, tissue, organs, nails or teeth. One embodiment includes ablation and/or blocking ablation by color reflected can be done. In one embodiment, includes 3-color or broadband-white illumination or with illumination that enhances color contrast (see, e.g., Thornton U.S. Pat. No. 3,963,953). In another embodiment, bases removal on the higher-temperature-indicating infrared light emitted by a surface. In another embodiment, a control input based total intensity (brightness) is used. In one embodiment, the control is through controlling on contrast and using a black-and-white video camera.

One embodiment includes location indicators placed on the surface wherein the system can adjust for some movement of the surface, and/or stop ablation upon excessive surface movement. The location markers can be outside the ablation area, but may also be inside the ablation area. In one embodiment, the markers located inside the ablation area are avoided by color or by inputting an ablation-free portion within the ablation area into the area specification. In another embodiment, an ablation-free portions within the ablation area may be inputted into the area specification for other reasons as well. In some embodiments, ablation area is defined by location indictors placed on the surface, e.g., by corners of a trapezoid.

In one embodiment, the system controls ablation only within a predetermined distance from a probe end, and can avoid ablation when the target is not in position and thus avoid accidents, (see “Altering The Emission Of An Ablation Beam for Safety or Control” provisional Docket No.ABI-15; which is incorporated by reference herein). Thus, the system can adjust for distance within a range and stop emission outside of that range. In one embodiment, the measurement can be measured sonically, by measuring backpressure from air-jet (or suction, or air-jet tube and suction tube combination), or by measuring a dimension of size of an auxiliary (e.g., conical) light beam.

One embodiment includes a method of controlling a ablation beam for ablation of a body surface in an environment isolated for safety and/or illumination reasons, including positioning a video camera to view the body surface in an isolated environment where the body surface needs to be in an isolated environment; providing a remotely-controllable ablation beam that can be controlled impinge at various locations on the surface and ablation can be controlled either directly by the control system or by using a monitor in a monitor-containing control module.

In some embodiments, the camera is of the “in-vivo” type (see “Camera Containing Medical Tool” provisional application No. 60/472,071; Docket No.ABI-4; filed May 20, 2003; which is incorporated by reference herein). In one embodiment, the camera can use optical fibers including one GRIN optical fiber which can maintain the scanning an externally scanned beam while conveying the scanning beam to the exit of the fiber and scans a surface, and one or more optical fibers to convey reflections back. One embodiment has 7 optical fibers including 1 GRIN and 6 to convey the reflections back. One embodiment synchronizes the scanning beam and the reflected signal back to a display. One embodiment includes a remote vidicon-containing camera body or a monitor. In another embodiment, an image can be displayed or information can be supplied to a control system. In one embodiment, the camera supplies its own illumination and can operate especially well with little or no ambient light environment. In another embodiment, a camera using wire or optical fiber to convey an image back from a probe to a remote camera body is used. Another embodiment includes a vidicon-containing camera with a GRIN fiber lens. In another embodiment, an endoscope type camera can be used.

One embodiment combines IR and visible cameras to provide alignment between the ablation beam and the camera. The IR camera (or IR portion of a dual IR-visible camera) can sense the small temperature change caused by the ablation and, e.g., place a beam-marker on the video display.

In some embodiments, the system allows ablation pulses only within predefined target parameters based on measurements, such as sonic feedback, or size of conical or cross-shaped auxiliary light beam with on target, or from backpressure of an air-jet (or air-jet and suction combination). In one embodiment, the parameters include predefined range of probe to target distance. In another embodiment, the system allows audible and/or visible indication of ablation pulses. In one embodiment, the indication is a color change of the auxiliary beam or a change in tone when ablation pulse is active.

In one embodiment, the environment is isolated for safety or illumination reasons by a curtain, or can be supplied at least in part by a shadow. In one embodiment, the curtain is black, blue, brown, gray, purple or other color. In another embodiment, the apparatus contains the probe positioner, wherein the apparatus is positioned over the surface being ablated. In one embodiment, the location is generally downward, including, e.g., 45° from vertical. In some embodiment, the apparatus containing the probe positioner also contains the camera, and in many embodiments contains an illumination source. In one embodiment, the ablating beam and illumination are emitting from below, and the body provides some of the illumination control. In one embodiment, the illumination control is for safety reasons. In one embodiment, light of 1550 nm is used. As used herein, the term “light” includes photons of wavelengths from UV, through the visible, and through the IR, and the term “color” includes relations of wavelengths in the UV, visible, and IR, and also contrasts in intensity of reflections between one surface area and another surface area.

Note that the color of the reflection is influenced by the illumination spectrum. One embodiment includes UV illumination and a UV camera, or IR illumination and an IR camera. In other embodiments, an IR camera can be used without illumination to sense temperature of portions of surfaces. In another embodiment, a combination IR and visible camera can be used to provide alignment between the ablation beam and the camera. One embodiment sense the small temperature change caused by the ablation using an IR camera (or IR portion of a dual IR-visible camera). In another embodiment, a beam-marker is placed on the video display.

In one embodiment, an auxiliary marker (e.g., a red or green laser beam) is used to indicate where on the surface the ablation beam is pointing. In one embodiment, the marker-laser beam is conducted onto the probe by an optical fiber. In another embodiment, the ablation beam and the marker-laser beam are conducted onto the probe by an optical fiber. In one embodiment, both the ablation beam and the marker laser beam come in through a single fiber as they are to be aligned. In another embodiment, the ablation-supplying fiber is a hollow fiber, and the marker beam can come in through the cladding of the hollow fiber. In yet another embodiment, separate fibers are used.

In one embodiment, the beam is amplified in a fiber amplifier and the blocking is by shutting off the current to the fiber-amplifiers pump diodes, and in another embodiment, the beam is amplified in a semiconductor optical amplifier and the blocking is by shutting off the current to the semiconductor optical amplifier. In another embodiment, the beam can be blocked by other ways, such insertion of an adsorbing material in the beam path.

In one embodiment, the ablation probe is mounted on an x-y-z-positioner, and the probe moved in the z-direction to follow surfaces that are not flat. In another embodiment, moving the beam without moving the probe scans smaller ablation areas. In one embodiment, the scanning is accomplished by beam deflecting mirrors mounted on piezoelectric actuators (see “Scanned Small Spot Ablation With A High-Rep-Rate” U.S. Provisional Patent Applications Ser. No. 60/471,972, Docket No.ABI-6; filed May 20, 2003; which is incorporated by reference herein). In one embodiment, the auxiliary beam is scanned. In one embodiment, the beam scanner positions the beam only over a defined color. In another embodiment, the system actuators scan over a larger region but with the ablation beam only enabled to ablate portions with the defined color and/or area.

In one embodiment, the control system controls pump diode current to control an amplifier to a predetermined temperature. In another embodiment, the repetition rate of the pulse generator is adjusted to control the pulse energy for efficient material removal. One embodiment includes one or more amplifiers used in a train mode (pulses from one amplifier being delayed to arrive one or more nanoseconds after those from another amplifier) allowing step-wise control of ablation rate independent of pulse energy.

In one embodiment, the distance from the surface to be ablated is measured sonically (measuring time between “ping” and receiving of the echo, much like sonar), and in another embodiment, the measurement is by measuring backpressure from air-jet (or suction induced pressure, or air-jet and suction combination) as a nearby object slows the flow, which raises the jet backpressure (or drop the pressure in the suction line). In either case, the beam can be blocked if no signal indicating a distance less than the maximum distance is received (see “Altering The Emission Of An Ablation Beam for Safety or Control” provisional application Docket No.ABI-15; which is incorporated by reference herein). In still another embodiment, a distance control system repositions the probe to maintain the probe in a predetermined range of distance from the surface being ablated. The system controls ablation within a predetermined distance from a probe end whereby avoiding ablation when the target is not in position, thus, avoiding accidents.

In one embodiment, the auxiliary beam is used to give a preliminary indication to an operator (e.g., a surgeon) where the ablation will take place. The auxiliary light beam may have a line or area shape. The auxiliary beam may be scanned (with the beam scan length controlled to be the same as the auxiliary light-beam length), whereby the length of a cut can be displayed before the ablation takes place. In another embodiment, the area of ablation can be displayed and controlled.

Typically, ablation has a threshold of less than 1 Joule per square centimeter, but occasionally surgical removal of foreign material may require dealing with an ablation threshold of up to about 2 Joules per square centimeter. In one embodiment, the system operates with pulses at about three times the ablation threshold for greater ablation efficiency. In one embodiment, the ablation rate is controllable independent of pulse energy. One embodiment includes one or more amplifiers used in a train mode (pulses from one amplifier being delayed to arrive one or more nanoseconds after those from another amplifier) allowing step-wise control of ablation rate independent of pulse energy. In another embodiment where lower ablation rates are needed, one or more amplifiers can be shut off (e.g., the optical pumping to the fiber amplifier shut off), whereby there will be fewer pulses per train. In one embodiment, 20 amplifiers are used producing a maximum of 20 pulses in a train. In another embodiment, three or four amplifiers and three or four pulses per train produced.

In one embodiment, the amplifiers are optically-pumped quasi-CW (pumping and amplifying perhaps 500 times per second in 1 millisecond bursts). In one embodiment, a Cr:YAG amplifier or fiber amplifier is used. One embodiment includes a quasi-CW, there is a pause between bursts, and the ratio of durations of the pause and the burst may be adjusted for component temperature and/or average repetition rate control. In another embodiment, non-CW-pumping is used in operating amplifiers, with amplifiers run in a staggered fashion, wherein one on for a first half-second period and then turned off for a second half-second period, and another amplifier, dormant during the first-period, turned on during the second period, and so forth, to spread the heat load.

Ablative material removal previously has been done using systems with optical benches weighing perhaps 1,000 pounds and occupying about 300 cubic feet or more. In one embodiment, the system can weigh 100 pounds or less and occupy 2.5 cubic feet or less. In some embodiments, the man-portable system comprises a cart and/or a backpack, in addition to the probe and connecting cables. One embodiment includes a combination of amplifier and small pulse-compressor enabling practical and significant size reduction. In another embodiment, the system is man-portable, including a wheeled cart and/or even in a backpack. A used herein, the term “man-portable” means capable of being moved reasonably easily by one person, e.g., as wheeling a wheeled cart from room to room or possibly even being carried in a backpack.

In one embodiment sub-picosecond pulses of between 10 picoseconds and one nanosecond are used, followed by pulse selection, with the selected pulses amplified by an amplifier (e.g., a erbium-doped fiber or Cr:YAG amplifier) and compressed by an air-path between gratings compressor (e.g., a Tracey grating compressor), with the compression creating a sub-picosecond ablation pulse. In another embodiment, a semiconductor oscillator to generate pulses and in some embodiments a SOA preamplifier is used to amplify the selected pulses before introduction into the amplifier.

One embodiment includes control of input optical signal power, optical pumping power of fiber amplifiers, timing of input pulses, length of input pulses, and timing between start of optical pumping and start of optical signals to control pulse power, and average degree of energy storage in fiber. In one embodiment, it is the pulse generator that controls the input repetition rate (which may be derived within the pulse generator from a higher repetition rate oscillator) of the amplifier to tune energy per pulse to about three times threshold per pulse.

One embodiment includes multiple moderate-power semiconductor-optical-amplifiers or optically pumped amplifiers, with the light pulse focused onto a very small area spot. In another embodiment, the system controls the amplifiers to give a pulse power controlled for optimum ablation efficiency. One embodiment measures light leakage from the delivery fiber to get a feedback proportional to pulse power and/or energy for control purposes. In one embodiment, the concentration of pulse energy on a small spot enables the use of semiconductor-optical amplifiers or moderate-power fiber-amplifiers. In another embodiment, the use of multiple moderate-power amplifiers allows ablation rate and pulse energy to be independently controlled, and also provides for more cost-effective, and more easily cooled, amplifiers. Thus, by the use of a combination of innovations, can now provide an efficient, reasonably-priced, man-portable ablation system for medical and other purposes. One embodiment includes measuring spot size with a video camera, with a stationary spot, or with a linear scan.

In one embodiment, moving the beam without moving the probe scans smaller ablation areas. In another embodiment, a large area may be scanned by moving the beam over a first area, and then stepping the probe to second portion of the large area and then scanning the beam over the second area, and so on. One embodiment includes scanning by beam deflecting mirrors mounted on piezoelectric actuators (see “Scanned Small Spot Ablation With A High-Rep-Rate” U.S. Provisional Patent Applications Ser. No. 60/471,972, Docket No.ABI-6; filed May 20, 2003; which is incorporated by reference herein). In another embodiment, the system actuators scan over a larger region but with the ablation beam only enabled to ablate portions with defined color and/or area. In one embodiment, a combination of time and, area and/or color, are preset to allow evaluation after a prescribed time.

Information of such a system and other information on ablation systems are given in co-pending provisional applications listed in the following paragraphs (which are also at least partially co-owned by, or exclusively licensed to, the owners hereof) and are hereby incorporated by reference herein (provisional applications listed by docket number, title and provisional number):
Docket No.ABI-1 Laser Machining U.S. Provisional Patent Applications Ser. No. 60/471,922; ABI-4 “Camera Containing Medical Tool” U.S. Provisional Patent Applications Ser. No. 60/472,071; ABI-6 “Scanned Small Spot Ablation With A High-Rep-Rate” U.S. Provisional Patent Applications Ser. No. 60/471,972; and ABI-7 “Stretched Optical Pulse Amplification and Compression”, U.S. Provisional Patent Applications Ser. No. 60/471,971, were filed May 20, 2003;
ABI-8 “Controlling Repetition Rate Of Fiber Amplifier” U.S. Provisional Patent Applications Ser. No. 60/494,102; ABI-9 “Controlling Pulse Energy Of A Fiber Amplifier By Controlling Pump Diode Current” U.S. Provisional Patent Applications Ser. No. 60/494,275; ABI-10 “Pulse Energy Adjustment For Changes In Ablation Spot Size” U.S. Provisional Patent Applications Ser. No. 60/494,274; ABI-11 “Ablative Material Removal With A Preset Removal Rate or Volume or Depth” U.S. Provisional Patent Applications Ser. No. 60/494,273; ABI-12 “Fiber Amplifier With A Time Between Pulses Of A Fraction Of The Storage Lifetime”; ABI-13 “Man-Portable Optical Ablation System” U.S. Provisional Patent Applications Ser. No. 60/494,321; ABI-14 “Controlling Temperature Of A Fiber Amplifier By Controlling Pump Diode Current” U.S. Provisional Patent Applications Ser. No. 60/494,322; ABI-15 “Altering The Emission Of An Ablation Beam for Safety or Control” U.S. Provisional Patent Applications Ser. No. 60/494,267; ABI-16 “Enabling Or Blocking The Emission Of An Ablation Beam Based On Color Of Target Area” U.S. Provisional Patent Applications Ser. No. 60/494,172; were filed Aug. 11, 2003. ABI-19 “High-Power-Optical-Amplifier Using A Number Of Spaced, Thin Slabs” U.S. Provisional Patent Applications Ser. No. 60/497,404 was filed Aug. 22, 2003;
Co-owned ABI-20 “Spiral-Laser On-A-Disc”, U.S. Provisional Patent Applications Ser. No. 60/502,879; and partially co-owned ABI-21 “Laser Beam Propagation in Air”, U.S. Provisional Patent Applications Ser. No. 60/502,886 were filed on Sep. 12, 2003. ABI-22 “Active Optical Compressor” U.S. Provisional Patent Applications Ser. No. 60/503,659 and ABI-23 “Controlling Optically-Pumped Optical Pulse Amplifiers” U.S. Provisional Patent Applications Ser. No. 60/503,578 were both filed Sep. 17, 2003;
ABI-24 “High Power SuperMode Laser Amplifier” U.S. Provisional Patent Applications Ser. No. 60/505,968 was filed Sep. 25, 2003, ABI-25 “Semiconductor Manufacturing Using Optical Ablation” U.S. Provisional Patent Applications Ser. No. 60/508,136 was filed Oct. 2, 2003, ABI-26 “Composite Cutting With Optical Ablation Technique” U.S. Provisional Patent Applications Ser. No. 60/510,855 was filed Oct. 14, 2003 and ABI-27 “Material Composition Analysis Using Optical Ablation”, U.S. Provisional Patent Applications Ser. No. 60/512,807 was filed Oct. 20, 2003;
ABI-28 “Quasi-Continuous Current in Optical Pulse Amplifier Systems” U.S. Provisional Patent Applications Ser. No. 60/529,425 and ABI-29 “Optical Pulse Stretching and Compressing” U.S. Provisional Patent Applications Ser. No. 60/529,443, were both filed Dec. 12, 2003;
   ABI-30 “Start-up Timing for Optical Ablation System” U.S. Provisional Patent Applications Ser. No. 60/539,026; ABI-31 “High-Frequency Ring Oscillator”, U.S. Provisional Patent Applications Ser. No. 60/539,024; and ABI-32 “Amplifying of High Energy Laser Pulses”, U.S. Provisional Patent Applications Ser. No. 60/539,025; were filed Jan. 23, 2004; and
ABI-33 “Semiconductor-Type Processing for Solid-State Lasers”, U.S. Provisional Patent Applications Ser. No. 60/543,086, was filed Feb. 9, 2004; and ABI-34 “Pulse Streaming of Optically-Pumped Amplifiers”, U.S. Provisional Patent Applications Ser. No. 60/546,065, was filed Feb. 18, 2004. ABI-35 “Pumping of Optically-Pumped Amplifiers”, was filed Feb. 26, 2004.

Although the present invention and its advantages have been described in detail, it should be understood that various changes, substitutions and alterations can be made herein without departing from the spirit and scope of the invention as defined by the appended claims. While use by a surgeon is an important use, the system can have other uses. Moreover, the scope of the present application is not intended to be limited to the particular embodiments of the process, machine, manufacture, composition of matter, means, methods and steps described in the specification, but only by the claims.

1. A method of controlling a ablation beam for ablation of a body surface, comprising:
positioning a video camera to view the body surface in an isolated environment whereby the body surface needs to be in an isolated environment because of contagious diseases, radiation, or special illumination control;
providing a remotely-controllable ablation beam that can be controlled to impinge at various locations on the surface and can be remotely blocked or enabled;
receiving a video signal from the video and displaying the signal on a video monitor; and
controlling the beam using a control module and the video monitor, wherein the control module and the video monitor are remote from the body surface.

2. The method of claim 1, wherein a surgeon uses a touch screen to control the ablation.

3. The method of claim 1, wherein the beam is controlled in position by an x-y table.

4. The method of claim 3, wherein the beam passes through a variable-focus lens on the x-y table and the variable-focus lens, and wherein the control module automatically adjusts the lens for changes in distance between the body surface and the table.

5. The method of claim 1, wherein the control module enables or blocks emission of the ablation beam based on colors of the target area, based on one or more preset emission specifications.

6. The method of claim 5, wherein the beam is amplified in an optically-pumped amplifier and the blocking is by shutting off the current to the amplifiers pump diodes or wherein the beam is amplified in a semiconductor optical amplifier and the blocking is by shutting off the current to the semiconductor optical amplifier.

7. The method of claim 5, wherein there is an over-ride switch that allows ablation to be enabled regardless of a blocking color specification.

8. The method of claim 1, wherein special illumination control is used and the measured color is either an IR color or a UV color.

9. The method of claim 1, wherein special illumination control is used and color contrast enhancing lighting is used.

10. The method of claim 1, wherein the wherein control module enables or blocks emission of the ablation beam based on colors of the target area and distance from a surface, based on preset emission specifications.

11. The method of claim 1, wherein the ablation-probe is mounted on a control-module-positionable table.

12. A method of controlling an ablation beam for ablation of a surface, comprising:
positioning at least one sensor to view the surface and sending a signal from the sensor to a remote control module;
providing a remotely-controllable ablation beam that can be controlled to impinge optical pulses ten picoseconds or less in duration at various locations on the surface; and
controlling location where the beam impinges the surface using a control module based on the signal received from the at least one sensor.

13. The method of claim 12, wherein control module enables or blocks emission of the ablation beam based on colors of the target area or distance from a surface, based on one or more preset emission specifications.

14. The method of claim 12, wherein the beam impingement is controlled by an x-y table.

15. The method of claim 14, wherein the x-y table also has a z-drive.

16. The method of claim 14, wherein the beam passes through a variable-focus lens on the x-y table and the variable-focus lens, and wherein the control module automatically adjusts the lens for changes in distance between the surface and the table.

17. The method of claim 12, wherein an operator uses a touch screen to control the ablation.

18. A method of controlling an ablation beam in the ablation of an object, comprising:
positioning at least one sensor to view the object;
providing a remotely-controllable ablation beam that can be controlled to impinge optical pulses of ten picoseconds or less in duration at various locations on the object; and
controlling the beam impingement using a control module that receives a signal from the at least one sensor, wherein the control module is remote from the object.

19. The method of claim 18, wherein the control module directs the ablation over a custom-shaped area.

20. The method of claim 18, wherein the control is remote for safety reasons.
